(19) United States
(12) Patent Application Publication
(10) Pub. No.: US 2018/0161552 A1
(43) Pub. Date: Jun. 14, 2018

(54) THROMBOLYSIS CATHETER SYSTEM

(71) Applicant: BOSTON SCIENTIFIC SCIMED, INC., Maple Grove, MN (US)

(72) Inventors: Kenneth R. Larson, Grand Rapids, MN (US); Meghan Flynn Prose, Minneapolis, MN (US)

(73) Assignee: BOSTON SCIENTIFIC SCIMED, INC., Maple Grove, MN (US)

(21) Appl. No.: 15/840,846

(22) Filed: Dec. 13, 2017

Related U.S. Application Data
(60) Provisional application No. 62/433,761, filed on Dec. 13, 2016.

(51) Int. Cl. A61M 25/10

(52) U.S. Cl. CPC A61M 25/1011; A61M 2025/1079; A61M 2025/1097; A61M 2025/105; A61M 2025/1052; A61M 2025/1015

(57) ABSTRACT

A catheter system is disclosed. An example catheter system includes an elongate tubular member having a distal region and a proximal end, a first balloon disposed along the distal region of the tubular member, a second balloon disposed along the distal region of the tubular member, wherein the second balloon is spaced from the first balloon. The catheter system also includes a first lumen extending within the elongate tubular member, a second lumen extending within the elongate tubular member, a first set of apertures in fluid communication with the first lumen, wherein the first set of apertures are configured to inflate both the first balloon and the second balloon and a second set of apertures in fluid communication with the first lumen, wherein the second set of apertures are configured to apply a treatment to a target site positioned between the first balloon and the second balloon.

### CROSS-REFERENCE TO RELATED APPLICATIONS

This application claims the benefit of priority under 35 U.S.C. § 119 to U.S. Provisional Application Ser. No. 62/433,761, filed Dec. 13, 2016, the entirety of which is incorporated herein by reference.

### TECHNICAL FIELD

The disclosure is directed to infusion catheters and accessory devices for use with catheters. More particularly, the disclosure is directed to devices to aid in removing or accelerating the removal of thrombus.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example catheter system includes:

an elongate tubular member having a distal region and a proximal end;

a first balloon disposed along the distal region of the tubular member;

a second balloon disposed along the distal region of the tubular member, wherein the second balloon is spaced from the first balloon;

a first lumen extending within the elongate tubular member;

a second lumen extending within the elongate tubular member;

a first set of apertures in fluid communication with the first lumen, wherein the first set of apertures are configured to inflate both the first balloon and the second balloon; and

a second set of apertures in fluid communication with the first lumen, wherein the second set of apertures are configured to apply a treatment to a target site positioned between the first balloon and the second balloon.

Alternatively or additionally to any of the embodiments above, further comprising a third set of apertures in communication with the second lumen, and wherein the third set of apertures are configured to communicate with a portion of a body lumen positioned proximal of the first balloon.

Alternatively or additionally to any of the embodiments above, wherein the first set of apertures are aligned with a chamber of the first balloon and a chamber of the second balloon.

Alternatively or additionally to any of the embodiments above, wherein the tubular member further includes an infusion region located between the first balloon and the second balloon, and wherein the second set of apertures are positioned along the infusion region.

Alternatively or additionally to any of the embodiments above, wherein the first balloon and the second balloon are configured to shift from an unexpanded configuration to an expanded configuration, and wherein the first balloon and second balloon both form a fluid tight seal along an inner surface of the body lumen while in the expanded configuration.

Alternatively or additionally to any of the embodiments above, wherein the second lumen is configured to permit a guidewire to extend therein.

Alternatively or additionally to any of the embodiments above, wherein the first lumen further includes an inner surface, an outer surface, and a wall extending therebetween, and wherein each of the first set of apertures and second set of apertures extends through the wall.

Alternatively or additionally to any of the embodiments above, wherein the second lumen further includes an inner surface, an outer surface, and a wall extending therebetween, and wherein each of the third set of apertures extends through the wall.

Alternatively or additionally to any of the embodiments above, wherein the second set of apertures are spaced equidistant around the outer surface of the tubular member.

Alternatively or additionally to any of the embodiments above, wherein the proximal end of the elongate tubular member is configured to releasably attach to a fluid infusion system.

Another example catheter system includes:

an elongate tubular member having a distal end and a proximal end;

a first balloon disposed along the distal end of the tubular member;

a second balloon disposed along the distal end of the tubular member, wherein the second balloon is spaced distally away from the first balloon;

a first lumen extending within the elongate tubular member; and

a second lumen extending within the elongate tubular member;

wherein the first lumen is in communication with the first balloon and the second balloon;

wherein the first lumen is in fluid communication with a target site positioned between the first balloon and the second balloon.

Alternatively or additionally to any of the embodiments above, wherein the second lumen is in fluid communication with a portion of a body lumen positioned proximal of the first balloon.

Alternatively or additionally to any of the embodiments above, wherein the first lumen is in communication with the first balloon and the second balloon via a first set of apertures disposed along the tubular member.

Alternatively or additionally to any of the embodiments above, wherein the first lumen is in communication with the target site via a second set of apertures disposed along the tubular member.

Alternatively or additionally to any of the embodiments above, further comprising a third set of apertures in communication with the second lumen, and wherein the third set of apertures are configured to communicate with a portion of a body lumen positioned proximal of the first balloon.

Alternatively or additionally to any of the embodiments above, wherein the tubular member further includes an infusion region located between the first balloon and the second balloon, and wherein the second set of apertures are positioned within the infusion region.

Alternatively or additionally to any of the embodiments above, wherein the first balloon and the second balloon are configured to shift from an unexpanded configuration to an expanded configuration, and wherein the first balloon and second balloon forms a fluid tight seal along an inner surface of the body lumen while in the expanded configuration.

Alternatively or additionally to any of the embodiments above, wherein the proximal end of the elongate tubular member is configured to releasably attach to a fluid infusion system.

An example method of treating a body lumen includes:

introducing an infusion catheter into a body lumen, the infusion catheter including:
an elongate tubular member having a distal region and a proximal end;
a first balloon disposed along the distal region of the tubular member;
a second balloon disposed along the distal region of the tubular member, wherein the second balloon is spaced from the first balloon;
a first lumen extending within the elongate tubular member;
a second lumen extending within the elongate tubular member;
a first set of apertures in fluid communication with the first lumen, wherein the first set of apertures are configured to inflate both the first balloon and the second balloon; and
a second set of apertures in fluid communication with the first lumen, wherein the second set of apertures are configured to apply a treatment to a target site positioned between the first balloon and the second balloon;

injecting a treatment into the first lumen, wherein injecting the treatment inflates the first balloon and the second balloon to form a fluid tight seal along an inner surface of the body lumen;

delivering the treatment to the target tissue site after while the first balloon and the second balloon maintain a fluid tight seal along an inner surface of the body lumen; and

perfusing blood through the second lumen coincident with delivering the treatment to the target tissue site.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example infusion catheter;

FIG. 2 illustrates the distal portion of the example infusion catheter of FIG. 1;

FIG. 3 illustrates a cross-section along line 3-3 of the infusion catheter of FIG. 2;

FIG. 4 illustrates a cross-section along line 4-4 of the infusion catheter of FIG. 2;

FIG. 5 illustrates a cross-section along line 5-5 of the infusion catheter of FIG. 2;

FIGS. 6-8 illustrate an example method for using an example infusion catheter.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Infusion catheters and systems may be used to remove thrombus, plaques, lesions, clots, etc. from veins or arteries. These devices may be effective to remove acute thrombus. For example, in some instances infusion systems may be utilized to infuse a treatment (e.g., lytic agents) adjacent a target site to soften and/or dissolve an area of thrombus. Further, in some instances lytic agents may be infused down the length of a catheter system and released out a series of holes adjacent the target site. However, in some instances the treatment may be released such that it not only treats the adjacent target site, but diffuses throughout the body. Therefore, in some instances it may be desirable to isolate the application of a treatment solution (e.g., lytic solution) to an area immediate the target site. Additionally, it may be desirable to design a catheter system that permits the perfusion of blood through the catheter (and, hence, the blood vessel) coincident with the application of the treatment to the target region. By isolating the treatment (e.g., lytic agent) to a particular region and allowing blood to maintain flow around the treatment site, improved treatment of target sites may be achieved. Example catheters and catheter systems which isolate treatment (e.g., lytic agent) to a particular region and allow blood to maintain flow around the treatment site are disclosed.

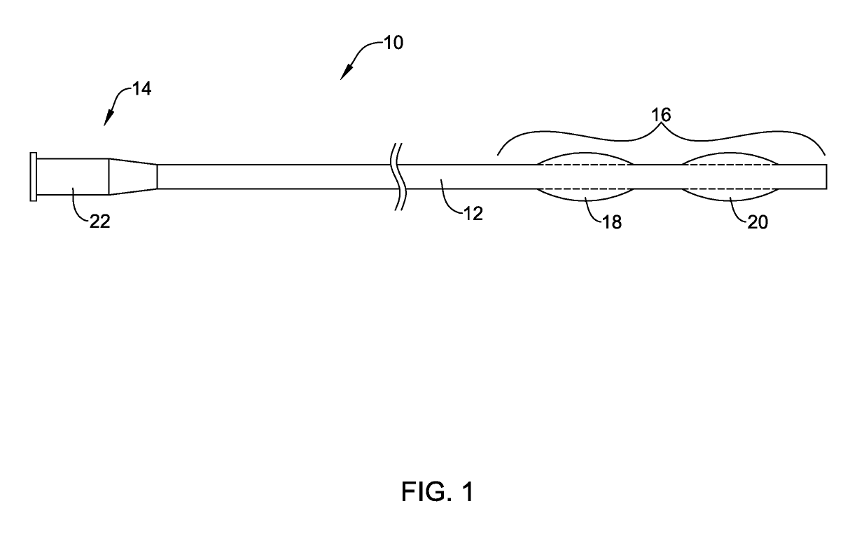

FIG. 1 shows an example infusion catheter 10. The infusion catheter 10 may include an elongated tubular member 12 extending between a proximal portion 14 and a distal portion 16 of the catheter 10. The infusion catheter 10 may include a first expandable member 18 and a second expandable member 20 disposed along the distal portion 16 of the catheter 10. In some examples, the second expandable member 20 may be spaced distal to the first expandable member 18. Further, FIG. 1 illustrates that the catheter 10 may include a hub 22 attached to the proximal end of the elongated tubular member 12 of the catheter 10.

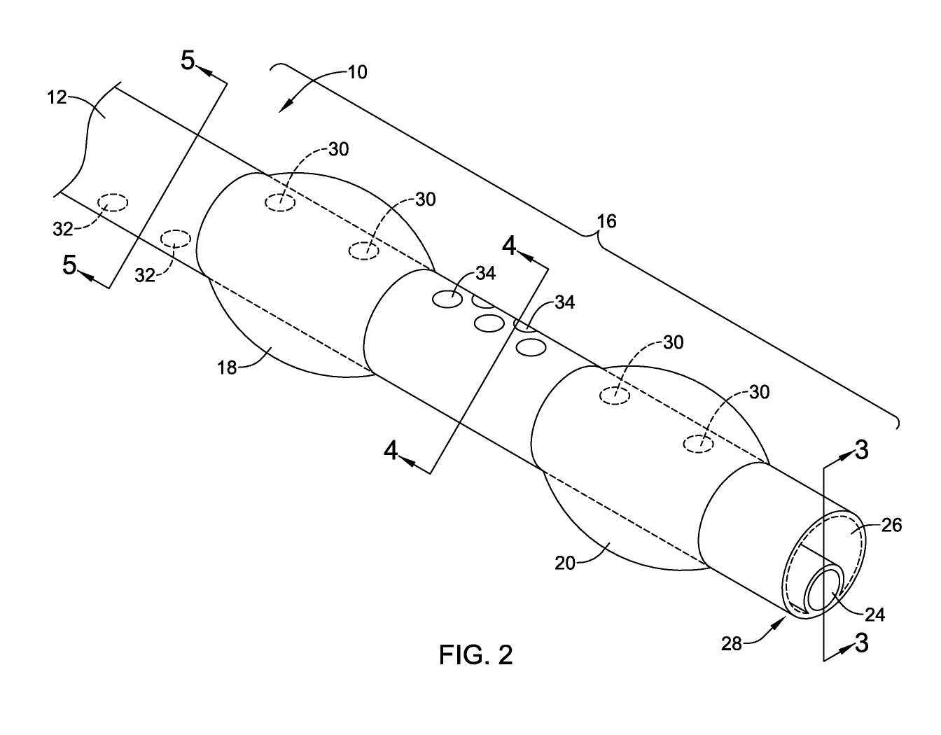

FIG. 2 illustrates the distal portion 16 of the catheter 10. As illustrated in FIG. 2, the tubular member 12 may include a first lumen 24 extending along a portion of or its entire length (e.g., the entire length of the tubular member 12 from the proximal portion 14 to the distal portion 16). The first lumen 24 may be designed to permit a guidewire (not shown in FIG. 2) to extend therein. The guidewire may be utilized to deliver the catheter 10 to a target treatment site. In other words, the catheter 10 may be advanced over a guidewire which has been previously placed adjacent a target site. It can be appreciated that the first lumen 24 may be designed such that a guidewire may extend through a hub 22, along the lumen 24 and eventually exiting the tubular member 12 at the distal end 28 of the distal portion 16.

Additionally, FIG. 2 illustrates that the tubular member 12 may include a second lumen 26 extending along a portion of or its entire length (e.g., the entire length of the tubular member 12 from the proximal portion 14 to the distal portion 16). As will be described in greater detail below, the lumen 26 of the tubular member 12 may be in fluid communication with a fluid infusion system attached to the hub member 22. The hub member 22 may be releasably attached to a fluid infusion system attached thereto.

In some examples, the lumen 26 of the tubular member 12 may not extend past the distal end 28 of the tubular member 12. For example, in some examples the lumen 26 extends substantially along the entire length of the catheter 10 (e.g., along the proximal portion 14 and the distal portion 16), but may stop short of the distal end 28 of the tubular member 12. FIG. 2 depicts the lumen 26 as a dashed line, signifying that the lumen is not open at the distal end of the tubular member 12. This design feature is important because it may be desirable for fluid that is passed along the lumen 26 to exit through one or more apertures located along the tubular member 12 rather than pass out the distal end of the tubular member 12 via the lumen 26.

FIG. 2 further illustrates that the catheter system 10 may include a first expandable member 18 and a second expandable member 20. It is noted that for purposes of the disclosure, the term expandable member may be used interchangeably with balloon. It can be appreciated that the first balloon 18 and the second balloon 20 may include a first unexpanded configuration (e.g., a wrapped configuration) and a second expanded configuration. The first balloon 18 and the second balloon 20 may be in an unexpanded configuration while positioned in a delivery system, for example. After being deployed from a delivery system, however, the first balloon 18 and the second balloon 20 may expand radially outward (e.g., away from the shaft member 12) to an expanded configuration.

As discussed above, FIG. 2 further shows a first set of apertures 30 disposed along the tubular member 12, a second set of apertures 34 disposed along the tubular member 12 and a third set of apertures 32 disposed along the tubular member 12. As will be discussed in greater detail below, the first set of apertures 30 and the second set of apertures 34 may be in fluid communication with the lumen 26 while the second set of apertures 32 may be in fluid communication with the lumen 24.

FIG. 2 further illustrates that the first set of apertures 30 may be aligned with the first balloon 18 and the second balloon 20. For example, FIG. 2 shows the apertures 30 positioned along the tubular member 12 such that they are in fluid communication with the interior chamber of the first expandable member 18 and the interior chamber of the second expandable member 20. In other words, the first balloon 18 and the second balloon 20 may be aligned along the tubular member 12 such that they cover (e.g., surround) the apertures 30.

Additionally, FIG. 2 illustrates that the second set of apertures 34 may be located along the tubular member 12 such that they are positioned between the first balloon 18 and the second balloon 20. Further, FIG. 2 illustrates that the third set of apertures 32 may be located along the tubular member 12 such that they are positioned proximal to the first balloon 18.

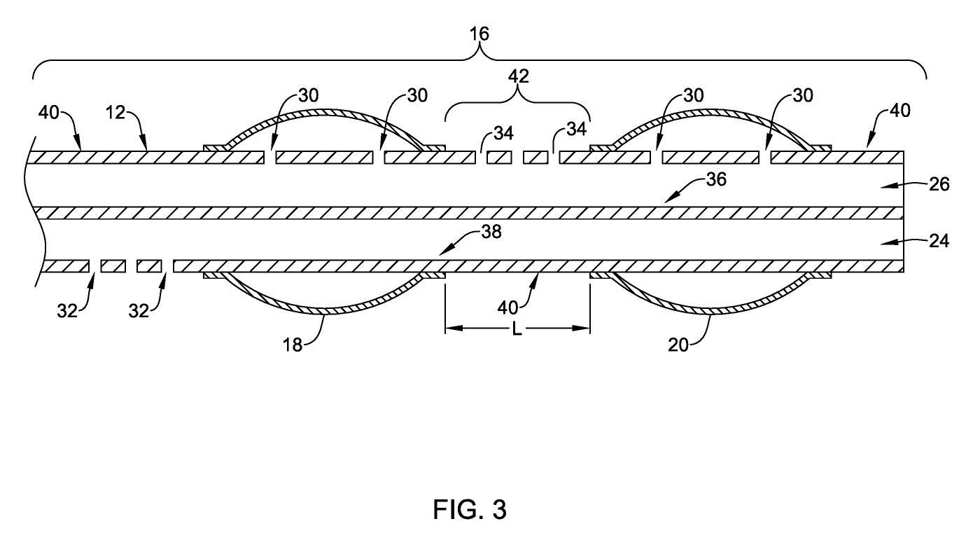

FIG. 3 illustrates a cross-section of distal portion 16 taken along line 3-3 of FIG. 2. Further, FIG. 3 illustrates that tubular member 12 may include a first, continuous inner surface 36 defining the lumen 26 of the tubular member 12. The tubular member 12 may also include an outer surface 40. Additionally, the tubular member 12 may include a tubular wall extending between the inner surface 36 of the lumen 26 and the outer surface 40 of the tubular member 12. It can further be appreciated that the one or more first apertures 30 and the one or more second apertures 34 may extend through the tubular wall. In other words, the first apertures 30 and/or the second apertures 34 may permit fluid communication between the lumen 26 of the tubular member 12 and the first balloon 18 (via the apertures 30), the second balloon 20 (via the apertures 30) and/or a body lumen (via the apertures 34) of the tubular member 12.

FIG. 3 further illustrates that the tubular member 12 may include a second, continuous inner surface 38 defining the lumen 24 of the tubular member 12. Additionally, the tubular member 12 may include a tubular wall extending between the inner surface 38 of the lumen 26 and the outer surface 40 of the tubular member 12. It can further be appreciated that one or more third apertures 32 may extend through the tubular wall defined between an inner surface 38 and an outer surface 40. In other words, the third set of apertures 32 may permit fluid communication between the lumen 24 of tubular member 12 and a body lumen in which catheter system is deployed, for example.

It can be appreciated from FIG. 3 that the first apertures 30 may be utilized to inflate the first balloon 18 and the second balloon 20. In some examples, the first balloon 18 and the second balloon 20 may be expanded via passing a fluid through the lumen 26 which, in turn, passes through the apertures 30 and into the first balloon 18 and the second balloon 20.

While FIG. 3 shows the apertures 30 as being spaced equidistant from each other, it is contemplated that the apertures 30 may be configured in a variety of patterns, arrangements, spacing, geometries, distributions, etc. In some examples, the apertures 30 may be unequal distances from one another.

Additionally, in some examples the apertures 30 may include more or fewer than the number of the apertures 30 depicted in FIG. 3. For example, each of the first balloon 18 and the second balloon 20 may be aligned with 1, 2, 3, 4, 5, 6, 7, 8, 9, 10, 25, 50, 75, 100 or more apertures 30, arranged in a variety of patterns. It can be further appreciated from FIG. 3 and the discussion above that each of the first apertures 30 illustrated in FIG. 3 may include an area, or opening, through which inflation fluid may flow via the lumen 26 of the tubular member 12 into each of the first balloon 18 and the second balloon 20.

For purposes of discussion herein, the “shape” of apertures 30 may be defined as the shape of the apertures 30 as outlined on the outer surface of the tubular member 12 in FIG. 3. For example, it can be appreciated that the “shape” of the apertures 30 shown in FIG. 3 is circular. In some examples, the “shape” of the apertures 30 may be referred to as the “cross-sectional shape” of the apertures 30. While the shape of the apertures 30 shown in FIG. 3 are depicted as circular, it is contemplated that the apertures 30 may be any shape or combinations of shapes. For example, the apertures 30 may be circular, rectangular, oval, triangular, square, etc. and combinations thereof. Further, the apertures 30 may have varying cross-sectional areas. For example, some of the apertures 30 may have a smaller cross-sectional area relative to other of the apertures 30, such that the rate of the inflation fluid passing through each of the apertures 30 may vary.

As will be discussed in greater detail below, third apertures 32 may be utilized to perfuse a fluid (e.g., blood, etc.) from a location proximal the first balloon 18 to location distal the second balloon 20. As can be appreciated form FIG. 3, the perfused fluid may travel (e.g., flow) from a position outside the lumen 12 (e.g., from inside a body lumen), through the guidewire lumen 24 via the apertures 32, to a position distal the distal end 28 of the tubular member 12.

While FIG. 3 shows the apertures 32 as being spaced equidistant from each other, it is contemplated that the apertures 32 may be configured in a variety of patterns, arrangements, spacing, geometries, distributions, etc. In some examples, the apertures 32 may be unequal distances from one another.

Additionally, in some examples the apertures 32 may include more or fewer than the number of the apertures 32 depicted in FIG. 3. For example, the apertures 32 may include 1, 2, 3, 4, 5, 6, 7, 8, 9, 10, 25, 50, 75, 100 or more apertures arranged in a variety of patterns. It can be further appreciated from FIG. 3 and the discussion above that each of the third apertures 32 illustrated in FIG. 3 may include an area, or opening, through which fluid may flow via the lumen 24 of the tubular member 12.

For purposes of discussion herein, the “shape” of the apertures 32 may be defined as the shape of the apertures 32 as outlined on the outer surface of the tubular member 12 in FIG. 3. For example, it can be appreciated that the “shape” of the apertures 32 shown in FIG. 3 is circular. In some examples, the “shape” of the apertures 32 may be referred to as the “cross-sectional shape” of the apertures 32. While the shape of the apertures 32 shown in FIG. 3 are depicted as circular, it is contemplated that the apertures 32 may be any shape or combinations of shapes. For example, the apertures 32 may be circular, rectangular, oval, triangular, square, etc. and combinations thereof. Further, the apertures 32 may have varying cross-sectional areas. For example, some of the apertures 30 may have a smaller cross-sectional area relative to other of the apertures 32, such that the rate of the fluid passing through each of the apertures 32 may vary.

As discussed above, in some instances it may be desirable to utilize the example infusion catheters disclosed herein to treat diseased tissue. For example, in some instances it may be desirable infuse a lytic agent into an occlusion present in a body lumen. Further, in some examples it may be desirable to infuse a lytic agent around a thrombus over a period of time. In other words, the examples disclosed herein may include infusion catheters in which a fluid (e.g., lytic agent) may soak or saturate a target site (e.g., thrombus) over a period of time. For example, the fluid may travel through the lumen 26 of the tubular member 12 to the apertures 34, whereby the fluid may pass from the lumen 26 of the tubular member 12, through the apertures 34 and surround diseased tissue.

In some instances, the one or more apertures 34 disclosed above may define an infusion region 42. For purposes of this disclosure, the infusion region 42 may define a region along the tubular member 12 within which the one or more apertures 34 may positioned. In some examples, the infusion region 42 may extend partially around the circumference of the tubular member 12. Further, the infusion region 42 may be defined along a given length along the tubular member 12. For example, FIG. 3 shows an infusion region 42 defined by the length “L” in FIG. 3. In other words, FIG. 3 defines an infusion region as the portion of the tubular member 12 in which the apertures 34 are located within length “L” and spaced around the outer circumference of the tubular member 12.

While FIG. 3 shows the apertures 34 as being spaced equidistant from each other, it is contemplated that the apertures 34 may be configured in a variety of patterns, arrangements, spacing, geometries, distributions, etc. In some examples, the apertures 34 may be unequal distances from one another.

Additionally, in some examples the apertures 34 may include more or fewer than the number of the apertures 34 depicted in FIG. 3. For example, an example infusion region 42 may include 1, 2, 3, 4, 5, 6, 7, 8, 9, 10, 15, 20, 30, 40, 50, 100 or more apertures 34 arranged in a variety of patterns.

It can be further appreciated from FIG. 3 and the discussion above that each of the apertures 34 illustrated in FIG. 3 may include an area, or opening, through which an infusion fluid may flow (via the lumen 26 of the tubular member 12). Further, it can be appreciated that the apertures 34, collectively, may define an area through which the infusion fluid may flow. The collective area through which fluid may flow for the apertures 34 may be calculated by adding the area of each individual aperture 34 located in the infusion region 42.

For example, the infusion region 42 may be defined as an area bound by the length “L” along the tubular member 12 and an arc length along the circumference of the tubular member 12. It can further be appreciated that this infusion region 42 may be represented (e.g., conceptualized) as a square have a surface area that is equal to the length “L” multiplied by the “arc length along circumference of tubular member” 12. Further, it can be appreciated that the area of the apertures 34 may be designed to provide an optimum flowrate of infusion fluid through the infusion region 42.

For purposes of discussion herein, the “shape” of the apertures 34 may be defined as the shape of the apertures 34 as outlined on the outer surface of the tubular member 12. For example, it can be appreciated that the “shape” of the apertures 34 shown in FIG. 2 is circular. In some examples, the “shape” of the apertures 34 may be referred to as the “cross-sectional shape” of the apertures 34. While the shape of the apertures 34 shown in FIG. 2 are depicted as circular, it is contemplated that the apertures 34 may be any shape or combinations of shapes. For example, the apertures 34 may be circular, rectangular, oval, triangular, square, etc. and combinations thereof.

Further, it can be appreciated that several design characteristics of the catheter 10 may directly affect the pressure, flow rate (e.g., velocity), energy, etc. at which a fluid may pass through the apertures 34. For example, it can be appreciated that the size (e.g., area), both individually and collectively, of the apertures 34 may contribute to the pressure, flow rate (e.g., velocity), energy, etc. at which a fluid may pass through the apertures 34. Additionally, the maximum infusion rate of the infusion system injecting fluid into the catheter 10 may directly affect the pressure, flow rate (e.g., velocity), energy, etc. at which a fluid may pass through the apertures 34. Specifically, the velocity and/or volume of fluid that passes through the apertures 34 may be a function of the total number of the apertures 34 present in the infusion zone 42, the area of each of the apertures 34 and the maximum infusion rate imparted by the system 10. Further, it can be appreciated that the aperture dimensions, aperture quantity, fluid flowrate, fluid pressure and the area of the infusion region are all variables which may be optimized for a given infusion system.

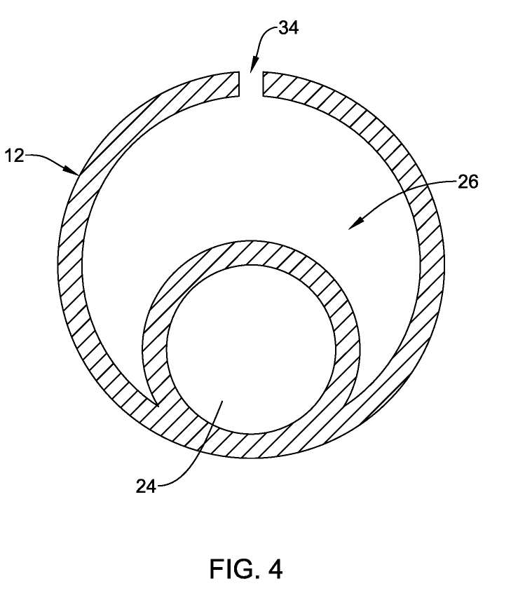

FIG. 4 illustrates a cross-section of the tubular member 12 along line 4-4 in FIG. 2. As illustrated in FIG. 4, the aperture 34 is in fluid communication with the lumen 26 of the tubular member 12. For example, FIG. 4 shows that an infusion fluid may flow from a position inside the lumen 26 to a location exterior to the lumen 26. Additionally, FIG. 4 shows the guidewire/perfusion lumen 24 being isolated from the infusion lumen 26. Additionally, FIG. 4 shows an example cross-sectional shape of the infusion lumen 26 and the guidewire/perfusion lumen 24. While the FIG. 4 shows the lumen 26 having a substantially crescent-shape and the lumen 24 having a circular shape, other cross-sectional shapes are contemplated. For example, the lumen 26 and/or the lumen 24 may be circular, rectangular, oval, triangular, square, etc.

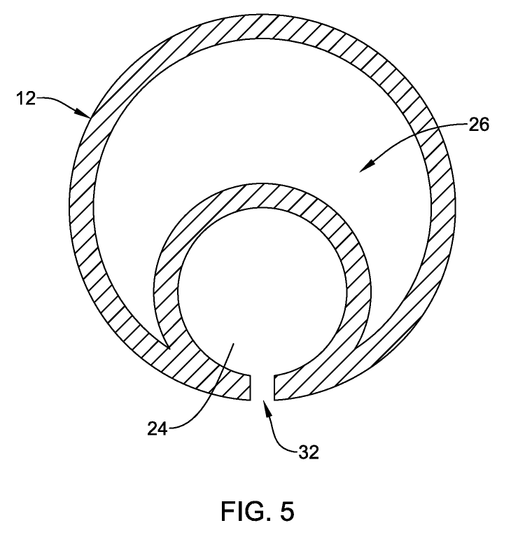

FIG. 5 illustrates a cross-section of the tubular member 12 along line 5-5 in FIG. 2. As illustrated in FIG. 5, the aperture 32 is in fluid communication with the lumen 24 of the tubular member 12. For example, FIG. 5 shows that a fluid (e.g., perfused blood) may flow from a position exterior to the lumen 24 to inside the lumen 24 (e.g., either traveling in a distal direction through the lumen 24 and out the distal end of the lumen 24 or in a proximal direction through the lumen 24 via the distal end of the lumen 24). Additionally, FIG. 5 shows the guidewire/perfusion lumen 24 being isolated from the infusion lumen 26. Additionally, FIG. 5 shows an example cross-sectional shape of the infusion lumen 26 and the guidewire/perfusion lumen 24. While the FIG. 5 shows the lumen 26 having a substantially crescent-shape and the lumen 24 having a circular shape, other cross-sectional shapes are contemplated. For example, the lumen 26 and/or the lumen 24 may be circular, rectangular, oval, triangular, square, etc.

As discussed above, in addition to applying the treatment solution (e.g., lytic) to the diseased tissue, it may be further desirable to isolate the target region of a body lumen for treatment. For example, it may be desirable to utilize the catheter system 10 to isolate (e.g., confine) a target region in a body lumen and infuse that particular region with a treatment (e.g., lytic) over a period of time.

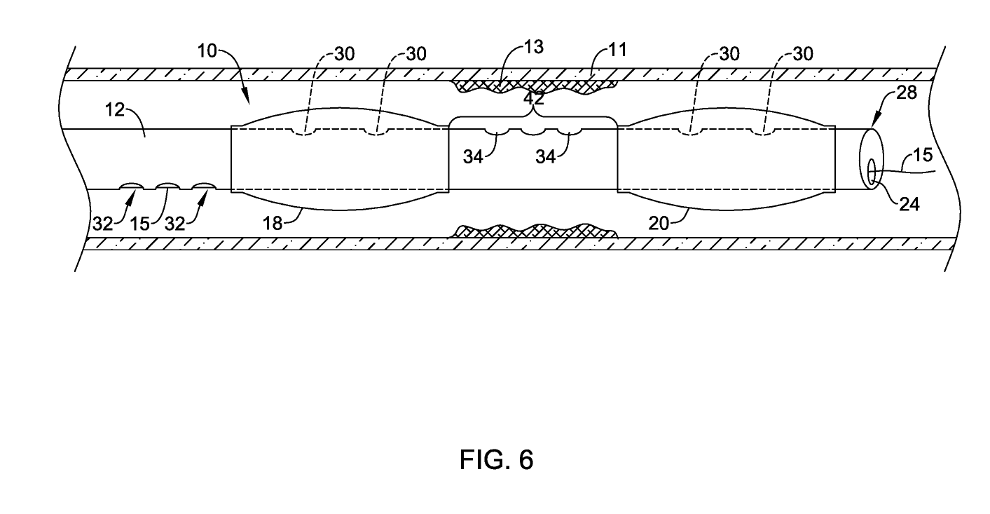

FIG. 6 illustrates an example first step in using the catheter 10 to isolate and treat a target site within an example body lumen. FIG. 6 shows the catheter system 10 positioned adjacent a target site 13 within a body lumen 11. Specifically, FIG. 6 shows the catheter 10 positioned such that the target site 13 is located between the first balloon 18 and the second balloon 20. Additionally, having the target site 13 positioned between the first balloon 18 and the second balloon 20 permits the infusion region 42 to be aligned (e.g., be positioned) adjacent the target site 13. Additionally, FIG. 6 shows the catheter system 10 being advanced over a guidewire 15. As discussed above, the guidewire 15 may extend within the lumen 24 of the tubular member 12.

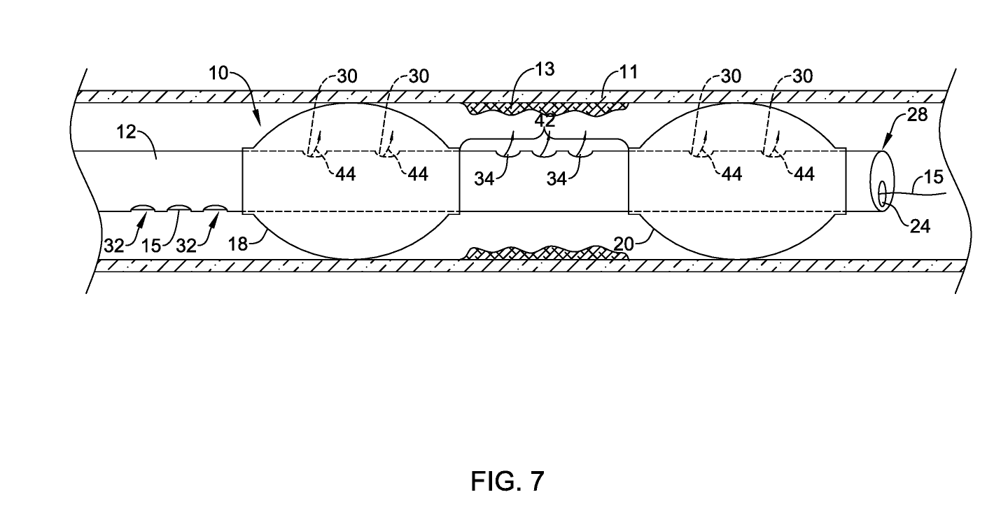

FIG. 7 illustrates an example second step in using the catheter 10 to isolate and treat a target site within an example body lumen. After the catheter 10 has been positioned within the body lumen 11 as described with respect to FIG. 6 above (e.g., such that target region 13 is located between the first balloon 18 and the second balloon 20 and the infusion region 42 is aligned with the target region 13), the first balloon 18 and the second balloon 20 may be inflated. In at least one example disclosed herein, the first balloon 18 and the second balloon 20 may be inflated using an inflation fluid passed through the lumen 26. It can be appreciated from the above discussion that the inflation fluid passed through the lumen 26 may be used to inflate both balloons and also pass the infusion fluid through the apertures 34 defining the infusion region 42.

For example, FIG. 7 shows dashed arrows 44 representing the infusion fluid (e.g., lytic agent) being passed through both the first set of apertures 30 to inflate the first balloon 18 and the second balloon 20. Additionally, FIG. 7 shows dashed arrows 44 representing the infusion fluid (e.g., lytic agent) being passed through the second set of apertures 34 to treat the target tissue 13. It can be appreciated that, in at least some examples, it may be desirable to initially permit a greater flowrate (e.g., volume) of infusion fluid to flow into the first balloon 18 and the second balloon 20 as compared to the fluid passing through the apertures 34 of the infusion region 42. As described above, it can be appreciated that it may be desirable to inflate the first balloon 18 and the second balloon 20 to an expanded configuration (and thereby isolate the target site 13), before a majority of the infusion fluid is passed through the infusion region 42. For example, FIG. 7 shows the first balloon 18 and the second balloon 20 inflated to an expanded configuration whereby they may form a tight, circumferential seal against the inner surface of the body lumen 11. It can be appreciated that these tight seals isolate and/or trap the infusion fluid (e.g., lytic agent) passing through the apertures 34 between the two balloons, thereby allowing the infusion fluid to surround and/or contact the target tissue until the first balloon 18 or the second balloon 20 is deflated.

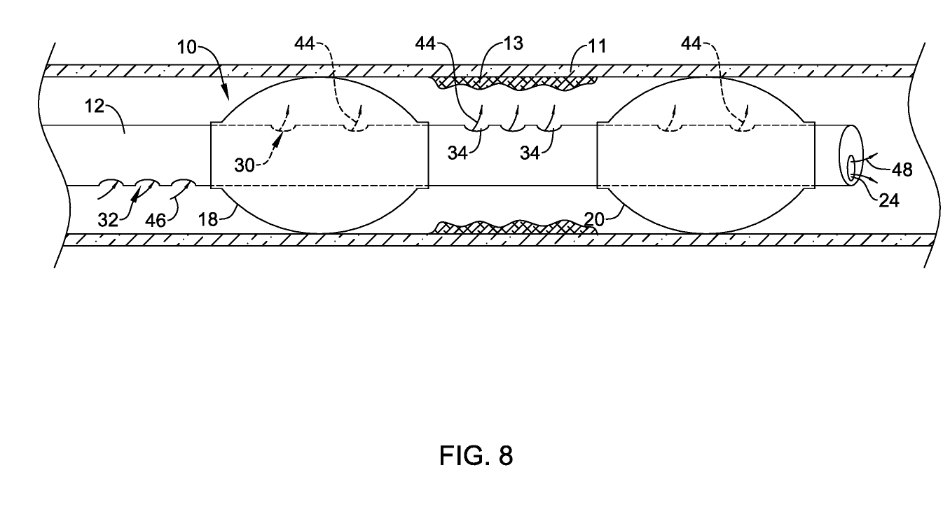

As discussed above, in some instances it may be desirable to permit blood located with the body lumen 11 to flow from a position proximal the first balloon 18 to a position distal to the second balloon 20 (however, this is not intended to be limiting, rather, in some examples it may be desirable to permit blood located with the body lumen 11 to flow proximally from a position distal the second balloon 20 to a position proximal the first balloon 18). Additionally, it may be desirable to perfuse blood through the lumen 24 while the first balloon 18 and the second balloon 20 are forming a fluid tight seal around the target tissue region 13. FIG. 8 shows the catheter 10 treating a target tissue site as described above in FIGS. 6 and 7. Further, the arrows 46 illustrate fluid (e.g., blood) entering the apertures 32 at a positioned proximal to the first balloon 18. It can be appreciated from the above discussion that after blood enters the apertures 32, it may travel in a distal direction through the lumen 24 and exit the lumen 24 (illustrated by the arrows 48) at the distal end 28 of the catheter 10. The perfusion of blood around the first balloon 18 and the second balloon 20 (while in the expanded configuration) may permit the catheter system 10 to treat the target tissue region 13 for an extended period of time.

Additionally, it is contemplated that in some instances the guidewire 15 may be repositioned within the lumen 24 as blood is perfused through the catheter system 10 (via the process described above). For example, FIG. 8 shows the guidewire 15 retracted to a positioned proximal the apertures 32 as blood is perfused through the lumen 24. As discussed, while not visible in FIG. 8, in some examples the guidewire 15 may remain in the lumen 24 while blood is perfused therethrough.

The catheter 10 or components thereof may be comprised of nickel-titanium alloy, stainless steel, a composite of nickel-titanium alloy and stainless steel, and/or include nickel-cobalt-chromium-molybdenum alloy (e.g., MP35-N). Alternatively, the catheter 10 or components thereof may be comprised of metals, polymers, combinations or composites thereof, or other suitable materials. In some instances, a portion or all of the catheter 10 or components thereof may be radiopaque to allow the catheter 10 or components thereof to be viewed on a fluoroscopy screen, or other imaging technique, during a procedure. In some instances, the distal end and/or coil may be radiopaque to aid the physician in determining the location of the distal end of the catheter 10 or components thereof.

The materials that can be used for the various components of the catheter 10 and/or other devices disclosed herein may include those commonly associated with medical devices. For simplicity purposes, the following discussion makes reference to accessory devices and their related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the accessory devices and their related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the accessory devices and their related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the accessory devices and their related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A catheter system, comprising:
an elongate tubular member having a distal region and a proximal end;
a first balloon disposed along the distal region of the tubular member;
a second balloon disposed along the distal region of the tubular member, wherein the second balloon is spaced from the first balloon;
a first lumen extending within the elongate tubular member;
a second lumen extending within the elongate tubular member;
a first set of apertures in fluid communication with the first lumen, wherein the first set of apertures are configured to inflate both the first balloon and the second balloon; and
a second set of apertures in fluid communication with the first lumen, wherein the second set of apertures are configured to apply a treatment to a target site positioned between the first balloon and the second balloon.

2. The catheter system of claim 1, further comprising a third set of apertures in communication with the second lumen, and wherein the third set of apertures are configured to communicate with a portion of a body lumen positioned proximal of the first balloon.

3. The catheter system of claim 1, wherein the first set of apertures are aligned with a chamber of the first balloon and a chamber of the second balloon.

4. The catheter system of claim 1, wherein the tubular member further includes an infusion region located between the first balloon and the second balloon, and wherein the second set of apertures are positioned along the infusion region.

5. The catheter system of claim 1, wherein the first balloon and the second balloon are configured to shift from an unexpanded configuration to an expanded configuration, and wherein the first balloon and second balloon both form a fluid tight seal along an inner surface of the body lumen while in the expanded configuration.

6. The catheter system of claim 1, wherein the second lumen is configured to permit a guidewire to extend therein.

7. The catheter system of claim 1, wherein the first lumen further includes an inner surface, an outer surface, and a wall extending therebetween, and wherein each of the first set of apertures and second set of apertures extends through the wall.

8. The catheter system of claim 2, wherein the second lumen further includes an inner surface, an outer surface, and a wall extending therebetween, and wherein each of the third set of apertures extends through the wall.

9. The catheter system of claim 1, wherein the second set of apertures are spaced equidistant around the outer surface of the tubular member.

10. The catheter system of claim 1, wherein the proximal end of the elongate tubular member is configured to releasably attach to a fluid infusion system.

11. An catheter system, comprising:
an elongate tubular member having a distal end and a proximal end;
a first balloon disposed along the distal end of the tubular member;
a second balloon disposed along the distal end of the tubular member, wherein the second balloon is spaced distally away from the first balloon;
a first lumen extending within the elongate tubular member; and
a second lumen extending within the elongate tubular member;
wherein the first lumen is in communication with the first balloon and the second balloon;
wherein the first lumen is in fluid communication with a target site positioned between the first balloon and the second balloon.

12. The catheter system of claim 11, wherein the second lumen is in fluid communication with a portion of a body lumen positioned proximal of the first balloon.

13. The catheter system of claim 11, wherein the first lumen is in communication with the first balloon and the second balloon via a first set of apertures disposed along the tubular member.

14. The catheter system of claim 13, wherein the first set of apertures are aligned with a chamber of the first balloon and a chamber of the second balloon.

15. The catheter system of claim 11, wherein the first lumen is in communication with the target site via a second set of apertures disposed along the tubular member.

16. The catheter system of claim 11, further comprising a third set of apertures in communication with the second lumen, and wherein the third set of apertures are configured to communicate with a portion of a body lumen positioned proximal of the first balloon.

17. The catheter system of claim 14, wherein the tubular member further includes an infusion region located between the first balloon and the second balloon, and wherein the second set of apertures are positioned within the infusion region.

18. The catheter system of claim 11, wherein the first balloon and the second balloon are configured to shift from an unexpanded configuration to an expanded configuration, and wherein the first balloon and second balloon forms a fluid tight seal along an inner surface of the body lumen while in the expanded configuration.

19. The catheter system of claim 11, wherein the proximal end of the elongate tubular member is configured to releasably attach to a fluid infusion system.

20. A method of treating a body lumen, the method comprising:
introducing an infusion catheter into a body lumen, the infusion catheter including:
an elongate tubular member having a distal region and a proximal end;
a first balloon disposed along the distal region of the tubular member;
a second balloon disposed along the distal region of the tubular member, wherein the second balloon is spaced from the first balloon;
a first lumen extending within the elongate tubular member;
a second lumen extending within the elongate tubular member;
a first set of apertures in fluid communication with the first lumen, wherein the first set of apertures are configured to inflate both the first balloon and the second balloon; and
a second set of apertures in fluid communication with the first lumen, wherein the second set of apertures are configured to apply a treatment to a target site positioned between the first balloon and the second balloon;
injecting a treatment into the first lumen, wherein injecting the treatment inflates the first balloon and the second balloon to form a fluid tight seal along an inner surface of the body lumen;
delivering the treatment to the target tissue site after while the first balloon and the second balloon maintain a fluid tight seal along an inner surface of the body lumen; and
perfusing blood through the second lumen coincident with delivering the treatment to the target tissue site.
